(19) United States
(12) Patent Application Publication
(10) Pub. No.: US 2007/0212331 A1
(43) Pub. Date: Sep. 13, 2007

(54) Methods and compositions for selectively killing cells

(76) Inventors: Joseph J. Baldassare, St. Louis, MO (US); Claudette I. Klein, St. Louis, MO (US)

Correspondence Address: SAINT LOUIS UNIVERSITY;OFFICE OF INNOVATION AND INTELLECTUAL PROPERTY, 3556 CAROLINE MALL, SUITE C208, ST. LOUIS, MO, 63104, US

(21) Appl. No.: 11/369,786

(22) Filed: Mar. 7, 2006

(51) Int. Cl. A61K 48/00; A61K 9/127; A61K 33/32

(52) U.S. Cl. 424/93.2; 424/450; 424/641; 514/6; 977/802; 977/907

(57) ABSTRACT

The inventors have discovered that increased levels of intracellular zinc leads to cell death by apoptosis and necrosis. Surprisingly, p53 minus cells under cell death when treated with zinc. Thus, the invention is directed to zinc compositions useful in the targeted delivery of zinc compounds to cancer cells, to kill the cancer cells. Such compositions may contain targeted liposomes as the delivery vehicle. Likewise, the invention is directed to methods of killing cancer cells as well as treating cancer in a patient. The method involved delivering zinc-containing compositions to a cancer cell.

### FIELD OF THE INVENTION

The invention relates to compositions and methods useful in the treatment of cell-based disease by the delivery of therapeutic amounts of a zinc compound. Specifically, the invention pertains to the treatment of cancer by the targeted introduction of zinc into cancer cells to induce cell death.

### BACKGROUND OF THE INVENTION

### Cancer

Cancer is a group of diseases characterized by uncontrolled growth and spread of abnormal cells. According to the American Cancer Society, in 2005 more than 570,280 Americans are expected to die of cancer. This equates to more than 1,500 people a day. Cancer is the second leading cause of death in the US, exceeded only by heart disease.

Generally, cancer is regarded as a disease of unchecked cell proliferation. Several cell biological processes, which relate either directly or indirectly to cell proliferation, have been discovered over the years that impinge carcinogenesis: (1) mitogen pathways and oncogenes, (2) cell cycle regulation and tumor suppressors, (3) growth factors and tumor promoters, and (4) programmed cell death. These pathways are well known and documented in the art. However, a brief synopsis of those pathways is presented.

### Oncogenes

The RAS/RAF/MEK/ERK signaling pathway is the prototypical mitogen activated protein (MAP) kinase pathway. In addition to being a mitogen pathway, it has been shown to affect a wide variety of cellular functions including cell proliferation, cell-cycle arrest, terminal differentiation and apoptosis. In general, a growth factor binds a membrane-bound receptor tyrosine kinase, which indirectly activates the small G-protein, RAS, which is embedded on the inner surface of the cell membrane. RAS is a branchpoint molecule in that it is capable of activating myriad cell signaling pathways. RAF is one such effecter of RAS. RAF is a cytosolic protein kinase, the first in a sequential chain of three such kinases, i.e., RAF-MEK-ERK. Once activated, RAF phosphorylates and activates MEK. MEK then phosphorylates and activates ERK, which in turn phosphorylates numerous other substrates and affects numerous cellular functions. Those functions include cellular differentiation, cell fate, proliferation, senescence and cell survival. Importantly, ERK signaling is disrupted in ˜30% of cancer cases (see Dumaz and Marais, FEBS Journal 272 (2005) pp. 3491-3504, which is herein incorporated by reference.)

### Cell Death

Cell death, which is a natural process, can be regulated through caspase-dependent (apoptosis) and caspase-independent mechanisms (apoptosis-like cell death, autophagic cell death or necrosis). Caspases are a class of cysteine proteases that are inactive as proenzymes and are activated by proteolytic maturation. Cell death can be stimulated by death receptor stimulation by death factors, such as tumor necrosis factor (TNF) and TRAIL, by cellular stress, and by DNA damage. For a review on cell death, see Kroemer and Martin, Nature Medicine 11(7) July 2005, pp. 725-730, which is herein incorporated by reference.

Apoptotic cells usually shrink and are phagocytised by other cells. Whereas necrotic cells (necrosis is another form of cell death) are characterized by swelling, especially of the mitochondria which become dysfunctional, followed by cell lysis.

### The Role of Zinc in Apoptosis and Homeostasis

It has been shown in the art that zinc can both induce and prevent apoptosis (see Fraker, P. and Telford, W. (1997) A reappraisal of the role of zinc in the life and death decision of life. Proc. Soc. Exp. Med. 215, 229-236, and Fraker, P. and Telford, W. (1996) Regulation of apoptotic events by zinc. In: Nutrition and Gene Expression (Berdanier, C., ed.), CRC Press, Boca Raton, FL, pp. 189-208, which are herein incorporated by reference.) It is taught therein that 80-200 μM zinc induced apoptosis in 40% of CD4+ CD8+ alpha beta TCR10CD3(10) thymocytes. According to the state-of-the art, zinc kills cells via apoptosis. Since a large number of cancer cells, which by their very nature lack the ability to undergo apoptosis, the art clearly teaches against using agents that kill cells through the induction of apoptosis. However, the inventors of the instant invention have been able to demonstrate that zinc not only kills by apoptosis, but also by another mechanism, namely necrosis. They have also been able to demonstrate the killing of p53 minus cells, which are not able to undergo canonical apoptosis. It is important to note that approximately 50% of tumors are p53 minus, which is attributed to the observation that they are resistant to apoptotic death.

Not only is zinc important in cell death, it has many other roles in the maintenance of cellular and organismal homeostasis. In general, zinc is required as either a catalytic, co-catalytic or structural component for a large number of enzymes, and thus contributes to a wide variety of important biological processes including gene expression, replication, hormonal storage and release, neurotransmission, and memory. A wide range of cells, e.g. neurons, pancreatic P cells, mammary and testicular cells, express zinc transporters to assure the uptake and the establishment of an appropriate intracellular concentration of zinc. It is also critical for the structural integrity of cells, influencing membrane stability and cytoskeletal organization. In this light, it is not surprising that dietary zinc deprivation is lethal in mice and has been associated in man with a variety of abnormalities related to growth, sexual maturation and wound healing. The complexity of zinc actions is further appreciated by the fact that the consequences of excess zinc are also severe, being associated with a number of neurodegenerative disorders including Parkinson's and Alzheimer's disease and epilepsy. Toward understanding the paradoxical functions of zinc, attention in the field has focused on defining the critical candidates that mediate its effect.

The immediate targets of the intracellular zinc are unclear, as are the physiological consequences of its actions on specific targets. Targets such as GADPH, NAD, and catabolizing activities such as poly(AFP-ribose) synthetase activity and production of ROS have been proposed as mediators of zinc neurotoxicity. However, zinc deprivation is also cytotoxic and there are numerous examples where culturing cells and zinc-deficient medium results in cell death while high external concentrations of zinc suppress that event. In that case, Ca2+/Mg2+ dependent endonucleases, capsases, Bcl2/Bax ratios, and cytoskeletal components are some of the proposed targets that Zn2+ modulates with a protective outcome. These examples indicate some of the difficulties in determining the critical candidates mediating the effects of zinc. Part of the difficulties arises from the lack of appropriate model systems that are amendable to molecular manipulation.

### Zinc as a Fixative in Tumor Excision

Zinc chloride has been used to fix tumors prior to surgical excision, but it has not been considered a chemotherapeutic agent in and of itself. Brooks et al. (U.S. Pat. No. 6,558,694, and references therein) describes a unit dose packaging of zinc chloride to be used as a tumor fixative prior to surgical excision. Prior to Brooks' innovation, Frederic Mohs (U.S. Pat. No. 2,344,830) taught the use of zinc chloride paste as a topical fixative to be applied prior to the surgical excision of melanomas and osteosarcomas. The inventors of the instant invention have made the surprising discovery that zinc compounds have cancer therapeutic activity, allowing for the first time the systemic (non-topical/non-fixative) delivery of zinc to cancer cells as part of a cancer therapeutic regimen.

### REFERENCES

The following references are cited through-out this disclosure to provide support to the invention. They are incorporated herein by reference. The inventors reserve the right to challenge the veracity of any statements therein.

1. Aizenman, E., A. K. Stout, K. A. Hartnett, K. E. Dineley, B. McLaughlin, and I. J. Reynolds. 2000. Induction of neuronal apoptosis by thiol oxidation: putative role of intracellular zinc release. J. Neurochem. 75:1878-1888.

2. Assaf, S. Y. and S. H. Chung. 1984. Release of endogenous Zn2+ from brain tissue during activity. Nature 308:734-736.

3. Azriel-Tamir, H., H. Sharir, B. Schwartz, and M. Hershfinkel. 2004. Extracellular zinc triggers ERK-dependent activation of Na+/H+ exchange in colonocytes mediated by the zinc-sensing receptor. J. Biol. Chem. 279:51804-51816.

4. Bataller, M. and J. Portugal. 2005. Apoptosis and cell recovery in response to oxidative stress in p53-deficient prostate carcinoma cells. Arch. Biochem. Biophys. 437:151-158.

5. Bhat, N. R. and P. Zhang. 1999. Hydrogen peroxide activation of multiple mitogen activated protein kinases in an oligodendrocyte cell line: role of extracellular signal regulated kinase in hydrogen peroxide-induced cell death. J. Neurochem. 72:112-119.

6. Bossy-Wetzel, E., M. V. Talantova, W. D. Lee, M. N. Scholzke, A. Harrop, E. Mathews, T. Gotz, J. Han, M. H. Ellisman, G. A. Perkins, and S. A. Lipton. 2004. Crosstalk between nitric oxide and zinc pathways to neuronal cell death involving mitochondrial dysfunction and p38-activated K+ channels. Neuron 41:351-365.

7. Chang, I., N. Cho, J. Y. Koh, and M. S. Lee. 2003. Pyruvate inhibits zinc-mediated pancreatic islet cell death and diabetes. Diabetologia 46:1220-1227.

8. Cho, S. G. and E. J. Choi. 2002. Apoptotic signaling pathways: caspases and stress activated protein kinases. J. Biochem. Mol. Biol. 35:24-27.

9. Choi, D. W. and J. Y. Koh. 1998. Zinc and brain injury. Annu. Rev. Neurosci. 21:347-375.

10. Clegg, M. S., L. A. Hanna, B. J. Niles, T. Y. Momma, and C. L. Keen. 2005. Zinc deficiency-induced cell death. IUBMB. Life 57:661-669.

11. Colucci-D'Amato, L., C. Perrone-Capano, and U. di Porzio. 2003. Chronic activation of ERK and neurodegenerative diseases. Bioessays 25:1085-1095.

12. Cuajungco, M. P. and G. J. Lees. 1997. Zinc metabolism in the brain: relevance to human neurodegenerative disorders. Neurobiol. Dis. 4:137-169.

13. Cullen, P. J. and P. J. Lockyer. 2002. Integration of calcium and Ras signalling. Nat. Rev. Mol. Cell Biol. 3:339-348.

14. Culmsee, C. and M. P. Mattson. 2005. p53 in neuronal apoptosis. Biochem. Biophys. Res. Commun. 331:761-777.

15. Eckhardt, S. 2002. Recent progress in the development of anticancer agents. Curr. Med. Chem. Anti.-Canc. Agents 2:419-439.

16. Erdal, H., M. Berndtsson, J. Castro, U. Brunk, M. C. Shoshan, and S. Linder. 2005. Induction of lysosomal membrane permeabilization by compounds that activate p53 independent apoptosis. Proc. Natl. Acad. Sci. U.S.A 102:192-197.

17. Fadeel, B. and S. Orrenius. 2005. Apoptosis: a basic biological phenomenon with wide ranging implications in human disease. J. Intern. Med. 258:479-517.

18. Feng, P., T. L. Li, Z. X. Guan, R. B. Franklin, and L. C. Costello. 2003. Effect of zinc on prostatic tumorigenicity in nude mice. Ann. N.Y. Acad. Sci. 1010:316-320.

19. Feng, P., J. Y. Liang, T. L. Li, Z. X. Guan, J. Zou, R. Franklin, and L. C. Costello. 2000. Zinc induces mitochondria apoptogenesis in prostate cells. Mol. Urol. 4:31-36.

20. Frederickson, C. J., S. W. Suh, D. Silva, C. J. Frederickson, and R. B. Thompson. 2000. Importance of zinc in the central nervous system: the zinc-containing neuron. J. Nutr. 130:1471S-1483S.

21. Geyer, M., C. Herrmann, S. Wohlgemuth, A. Wittinghofer, and H. R. Kalbitzer. 1997. Structure of the Ras-binding domain of RalGEF and implications for Ras binding and signalling. Nat. Struct. Biol. 4:694-699.

22. Haase, H., W. Watjen, and D. Beyersmann. 2001. Zinc induces apoptosis that can be suppressed by lanthanum in C6 rat glioma cells. Biol. Chem. 382:1227-1234.

23. Hancock, J. F. 2003. Ras proteins: different signals from different locations. Nat. Rev. Mol. Cell Biol. 4:373-384.

24. Herrmann, C., G. Horn, M. Spaargaren, and A. Wittinghofer. 1996. Differential interaction of the ras family GTP-binding proteins H-Ras, Rap1A, and R-Ras with the putative effector molecules Raf kinase and Ral-guanine nucleotide exchange factor. J. Biol. Chem. 271:6794-6800.

25. Isoldi, M. C., M. A. Visconti, and A. M. Lauro Castrucci. 2005. Anti-cancer drugs: molecular mechanisms of action. Mini. Rev. Med. Chem. 5:685-695.

26. Jansen, S., J. Arning, and D. Beyersmann. 2003. Effects of the Ca ionophore a23187 on zinc-induced apoptosis in C6 glioma cells. Biol. Trace Elem. Res. 96:133-142.

27. Jiang, D., P. G. Sullivan, S. L. Sensi, 0. Steward, and J. H. Weiss. 2001. Zn(2+) induces permeability transition pore opening and release of pro-apoptotic peptides from neuronal mitochondria. J. Biol. Chem. 276:47524-47529.

28. Kane, D. J., T. A. Sarafian, R. Anton, H. Hahn, E. B. Gralla, J. S. Valentine, T. Ord, and D. E. Bredesen. 1993. Bcl-2 inhibition of neural death: decreased generation of reactive oxygen species. Science 262:1274-1277.

29. Kawahara, M., M. Kato-Negishi, and Y. Kuroda. 2002. Pyruvate blocks zinc-induced neurotoxicity in immortalized hypothalamic neurons. Cell Mol. Neurobiol. 22:87-93.

30. Kim, B. J., Y. H. Kim, S. Kim, J. W. Kim, J. Y. Koh, S. H. Oh, M. K. Lee, K. W. Kim, and M. S. Lee. 2000. Zinc as a paracrine effector in pancreatic islet cell death. Diabetes 49:367-372.

31. Koh, J. Y. 2001. Zinc and disease of the brain. Mol. Neurobiol. 24:99-106.

32. Kondoh, M., E. Tasaki, S. Araragi, M. Takiguchi, M. Higashimoto, Y. Watanabe, and M. Sato. 2002. Requirement of caspase and p38MAPK activation in zinc-induced apoptosis in human leukemia HL-60 cells. Eur. J. Biochem. 269:6204-6211.

33. Kroemer, G., B. Dallaporta, and M. Resche-Rigon. 1998. The mitochondrial death/life regulator in apoptosis and necrosis. Annu. Rev. Physiol 60:619-642.

34. Lobner, D., L. M. Canzoniero, P. Manzerra, F. Gottron, H. Ying, M. Knudson, M. Tian, L. L. Dugan, G. A. Kerchner, C. T. Sheline, S. J. Korsmeyer, and D. W. Choi. 2000. Zinc-induced neuronal death in cortical neurons. Cell Mol. Biol. (Noisy.-le-grand) 46:797-806.

35. MacPherson, D., J. Kim, T. Kim, B. K. Rhee, C. T. Van Oostrom, R. A. DiTullio, M. Venere, T. D. Halazonetis, R. Bronson, A. De Vries, M. Fleming, and T. Jacks. 2004. Defective apoptosis and B-cell lymphomas in mice with p53 point mutation at Ser 23. EMBO J. 23:3689-3699.

36. Malumbres, M. and M. Barbacid. 2003. RAS oncogenes: the first 30 years. Nat. Rev. Cancer 3:459-465.

37. Mann, J. J. and P. J. Fraker. 2005. Zinc pyrithione induces apoptosis and increases expression of Bim. Apoptosis. 10:369-379.

38. McDermott, U., D. B. Longley, L. Galligan, W. Allen, T. Wilson, and P. G. Johnston. 2005. Effect of p53 status and STAT1 on chemotherapy-induced, Fas-mediated apoptosis in colorectal cancer. Cancer Res. 65:8951-8960.

39. Mocchegiani, E., C. Bertoni-Freddari, F. Marcellini, and M. Malavolta. 2005. Brain, aging and neurodegeneration: role of zinc ion availability. Prog. Neurobiol. 75:367-390.

40. Nelson, D. A. and E. White. 2004. Exploiting different ways to die. Genes Dev. 18:1223-1226.

41. Nguyen, T. T., E. Tran, T. H. Nguyen, P. T. Do, T. H. Huynh, and H. Huynh. 2004. The role of activated MEK-ERK pathway in quercetin-induced growth inhibition and apoptosis in A549 lung cancer cells. Carcinogenesis 25:647-659.

42. Prasad, A. S. 1983. Zinc deficiency in human subjects. Prog. Clin. Biol. Res. 129:1-33.

43. Prasad, A. S. 1985. Clinical, endocrinological and biochemical effects of zinc deficiency. Clin. Endocrinol. Metab 14:567-589.

44. Rodriguez, I. R., S. Alam, and J. W. Lee. 2004. Cytotoxicity of oxidized low-density lipoprotein in cultured RPE cells is dependent on the formation of 7-ketocholesterol. Invest Ophthalmol. Vis. Sci. 45:2830-2837.

45. Samet, J. M., B. J. Dewar, W. Wu, and L. M. Graves. 2003. Mechanisms of Zn(2+)induced signal initiation through the epidermal growth factor receptor. Toxicol. Appl. Pharmacol. 191:86-93.

46. Seo, S. R., S. A. Chong, S. I. Lee, J. Y. Sung, Y. S. Ahn, K. C. Chung, and J. T. Seo. 2001. Zn2+-induced ERK activation mediated by reactive oxygen species causes cell death in differentiated PC12 cells. J. Neurochem. 78:600-610.

47. Sever, L. E. 1973. Zinc deficiency in man. Lancet 1:887.

48. Shimizu, S., Y. Eguchi, W. Kamiike, S. Waguri, Y. Uchiyama, H. Matsuda, and Y. Tsujimoto. 1996. Bcl-2 blocks loss of mitochondrial membrane potential while ICE inhibitors act at a different step during inhibition of death induced by respiratory chain inhibitors. Oncogene 13:21-29.

49. Soussi, T. 2003. p53 mutations and resistance to chemotherapy: A stab in the back for p73. Cancer Cell 3:303-305.

50. Soussi, T. and G. Lozano. 2005. p53 mutation heterogeneity in cancer. Biochem. Biophys. Res. Commun. 331:834-842.

51. Subramaniam, S., U. Zirrgiebel, H. 0. Bohlen Und, J. Strelau, C. Laliberte, D. R. Kaplan, and K. Unsicker. 2004. ERK activation promotes neuronal degeneration predominantly through plasma membrane damage and independently of caspase-3. J. Cell Biol. 165:357-369.

52. Truong-Tran, A. Q., L. H. Ho, F. Chai, and P. D. Zalewski. 2000. Cellular zinc fluxes and the regulation of apoptosis/gene-directed cell death. J. Nutr. 130:1459S-1466S.

53. Vallee, B. L. and K. H. Falchuk. 1993. The biochemical basis of zinc physiology. Physiol Rev. 73:79-118.

54. Vermeulen, K., D. R. Van Bockstaele, and Z. N. Berneman. 2005. Apoptosis: mechanisms and relevance in cancer. Ann. Hematol. 84:627-639.

55. Villa, E., I and R. M. Cunha Ferreira. 1985. Zinc, pregnancy and parturition. Acta Paediatr. Scand. Suppl 319:150-157.

56. Vojtek, A. B. and C. J. Der. 1998. Increasing complexity of the Ras signaling pathway. J. Biol. Chem. 273:19925-19928.

57. Wada, T. and J. M. Penninger. 2004. Mitogen-activated protein kinases in apoptosis regulation. Oncogene 23:2838-2849.

58. Wang, X., J. L. Martindale, and N. J. Holbrook. 2000. Requirement for ERK activation in cisplatin-induced apoptosis. J. Biol. Chem. 275:39435-39443.

59. Zhu, L., X. Yu, Y. Akatsuka, J. A. Cooper, and C. Anasetti. 1999. Role of mitogens activated protein kinases in activation-induced apoptosis of T cells. Immunology 97:26-35.

60. Zong, W. X., D. Ditsworth, D. E. Bauer, Z. Q. Wang, and C. B. Thompson. 2004. Alkylating DNA damage stimulates a regulated form of necrotic cell death. Genes Dev. 18:1272-1282.

### SUMMARY OF THE INVENTION

The inventors have made the surprising discovery that both cellular apoptosis and necrosis can be induced by increasing the intracellular concentration of zinc above a certain threshold.

Furthermore, by selectively targeting specific cells or specific types of cells, and delivering killing-effective amounts of zinc to those cells, those cells can be killed. This invention is particularly useful in treating patients suffering from cellular based diseases, such as cancer, whereby targeted-zinc is administered to the patient.

Thus the invention is directed to compositions and methods of killing cells by increasing the intracellular concentration of zinc to a level by which apoptosis and necrosis occurs in the cell. Preferably, but not necessarily exclusively, the cell to be killed is a cancer cell, which can be ex vivo or in vivo. As used herein, ex vivo broadly means cells in culture. Those cells can be directly from a patient or not. As used herein, in vivo means cells in a patient, wherein the patient broadly includes animals, more preferably humans. In the in vivo aspect, the invention is directed to compositions and methods for treating cancer in a patient.

Thus, in one embodiment, the invention is directed to a composition comprising an amount of zinc that is effective in killing a cell, wherein a preferred cell is a cancer cell. In a preferred embodiment, the composition comprises zinc encased by, associated with or linked to a vector for carrying the zinc and delivering the zinc to the cells. A currently preferred vector is a liposome, but other vectors may be used or developed in the future, which are applicable to the practice of this invention. Most preferably, this zinc-liposome complex is linked to a targeting moiety (infra), which enables the zinc-liposome complex to selectively bind a particular cell of interest, e.g., cancer cell. Targeting moieties can be, but not necessarily limited to ligands, cognate receptors, antibodies, aptamers and/or fragments thereof, e.g., for breast cancer the targeting moiety may be an antibody or antibody fragment that recognizes the Her-2 antigen. The zinc may be in any form. Current examples show the utility of zinc chloride, but the inventors envision that other zinc compounds may have more acceptable or even favorable features.

In yet another embodiment, the invention is directed to a method of killing a cell—again, preferably a cancer cell ex vivo or in vivo—comprising the step of delivering a therapeutic amount of zinc into the cell. Preferably, the zinc is encased in a vector, such as a liposome. More preferably, the vector contains a targeting moiety as set forth in the above embodiment (supra). In a preferred aspect of this embodiment, the method involves the preparation of a targeted zinc vector composition. For example, zinc (preferably a zinc salt) is incorporated utilizing a vector to wrap or otherwise entrap/contain the zinc.

In yet another embodiment, the invention is directed to a method for treating cancer, the method comprising the step of administering a zinc-targeted vector composition in a pharmaceutically acceptable form. The composition can be administered in any one or more acceptable means, such as intratumoral injection, topical administration to a tumor, intravenous, intraperitoneal, intranasal, by inhalation, and/or orally. The instant composition may be used alone or in combination with one or more cancer therapies, such as surgery, radiation and/or chemotherapy. The combination therapy may be synergistic, allowing for more effective treatment using lower doses of radiation and/or chemotherapeutic agents. The effect of the treatment is expected to be the arrest of the growth of the tumor, a reduction in the growth rate of the tumor, or regression of the tumor, relative to controls or untreated patients.

### BRIEF DESCRIPTION OF THE DRAWINGS

FIG. 1 depicts zinc-induced cell death in a time and dose dependent manner. (A) IIC9 cells (embryonic fibroblasts) were serum starved for 2 days and treated with the indicated concentrations of zinc for the indicated time periods. Cell death was monitored by trypan blue exclusion as described in the Materials and Methods. (B-D) Zinc-induced cell death is accompanied by morphological changes associated with apoptosis and necrosis. Shown are cells that were incubated with either 50 μM (C) or 200 μM (D) zinc for 6 hours. Control cells (B) were untreated. Cells were examined microscopically for changes in cell shape and size. Control cells retain the normal morphology while cells treated with 50 μM zinc were rounded and displayed differing degrees of cell shrinkage. Cells treated with 200 μM zinc showed a more varied morphology. Some cells were rounded, while others were somewhat enlarged and showed significant expansion of the cytosol (see arrows). Cell debris, indicative of cell lysis, was evident.

FIG. 2 demonstrates that zinc-induced cell death is ERK dependent. IIC9 cells were preincubated with U0126 (ERK inhibitor) SB203580 (p38 inhibitor) or SP600125 (JNK inhibitor) for 30 min. afterwhich they were treated with 50 μM zinc for the indicated time periods. Cell death was significantly reduced when ERK activation was prevented but not when p38 or JNK were inhibited. Incubation of cells with U0126 (D) also prevented the morphological changes induced by zinc (C). Control cells are shown in B. FIG. 2E shows that zinc does induce pERK, as determined by western blotting with pERK antibody (lane 1). The activation is significantly reduced when cells are preincubated with U0126 (lane 2).

FIG. 3 demonstrates that zinc mediated cell death is sensitive to inhibitors of apoptosis. Cells were preincubated with Z-VAD, a general caspase inhibitor and cell death monitored in response to the indicated concentrations of zinc. Z-VAD greatly attenuated death in the early hours (3-6) but its effects were less dramatic with longer incubation times and the higher zinc concentrations. FIG. 3B-D show control cells, cells treated with 50 μM zinc or cells treated with 50 μM zinc that had been transfected to over-express Bcl-XL. Over-expression attenuates zinc-mediated cell death and limits the morphological changes that accompany that death.

FIG. 4 demonstrates that zinc functions intracellularly. (A) IIC9 cells were preincubated with PD153035 (EGF receptor inhibitor) for 30 min. prior to treatment with either EGF or zinc, as indicated. ERK activation was determined by western blotting with pERK antibody. (B) Cells were preincubated with or without pyrithione for 30 min and then treated with the indicated concentrations of zinc. Cell death was monitored after a 6 h period. (C). Cells were preincubated with pyrithione and then treated for 1 h with 30 μM zinc (land 1) and monitored for pERK levels by western blotting. Levels of pERK in cells that had not been treated with pyrithione are shown in lane 2.

FIG. 5 depicts the activation of Ras by zinc. (A) IIC9 cells were treated with 200 μM ZnCl₂ for 1 h and then assayed for Ras activation using the RBD-pull down assay as described below in example 3. Untreated cells were also monitored. (B) Cells were transfected to over express dn-(dominant negative)Ras and then treated with 100 μM zinc for 6 h. Cell death was monitored by trypan blue exclusion as described in below in example 3.

FIG. 6 depicts the killing of PC3 cells by contacting the cells with liposome-encapsulated zinc chloride, non-encapsulated zinc, and targeted liposome-encapsulated zinc. Panel A depicts zinc treated PC3s, no liposome, no targeting moiety; note that the cells underwent cell death. Panel B depicts untreated PC3s, in which no cell death is observed. Panel C depicts PC3s with downregulated transferrin, no treatment (control for Panel E); note that the cells fail to undergo cell death. Panel D depicts PC3s treated with transferrin-tagged liposomes without zinc (control for panel F); note that the cells failed to undergo cell death. Panel E depicts PC3s with downregulated transferrin receptor exposed to transferrin-tagged liposomes containing zinc; note that the cells failed to undergo cell death. Panel F depicts PC3s treated with transferrin-tagged liposomes containing zinc; note that the cells underwent cell death. Panel G depicts PC3s treated with untagged liposomes containing zinc; note that the cells failed to undergo cell death.

### DETAILED DESCRIPTION OF PREFERRED EMBODIMENTS

The inventors have made the surprising discovery that cells, particularly cancer cells, die from overexposure to zinc. While it is known in the art that zinc overabundance or underabundance can induce or stimulate cells to undergo p53-mediated apoptosis, the inventors have successfully demonstrated that an increase in intracellular zinc beyond threshold levels required for homeostasis can cause cell death in both cells that express p53 and cells that are p-53 minus. This becomes important in the treatment of cancer, since most cancers have lost normal p53 activity, and therefore are unresponsive to chemotherapies that target p53-mediated activities. While not wishing to be bound by theory, this newly discovered activity of zinc allows for the use of zinc as a cancer therapeutic agent.

This discovery has lead to the instant invention, which includes compositions and methods useful in the treatment of cell-based diseases like cancer. The composition comprises a zinc compound, a targeting moiety, which allows for the composition to be targeted to the appropriate cell type, and a delivery vector, which carries and delivers the zinc compound to the appropriate cell type.

The inventors envision, and the skilled artisan would reasonably recognize, that any zinc compound will work in the practice of this invention. Exemplary zinc compounds include zinc chloride, zinc sulfate, zinc chelates, atomic zinc, and the like. Zinc chloride was used primarily in the experimental protocols, although ZnSO₄ was shown to be equally effective. The skilled artisan would likely say that different zinc compounds will have different potencies depending on the dissociation constant of the zinc-ligand complex.

The inventors envision that the delivery vector can be any vehicle that enables the zinc to be delivered intracellularly. For example, the delivery vector may be a liposome, a nanocarrier, a colloidal gold particle, a peptide, virus particle, intracellular bacteria, vesicle, cyclodextrin microsphere, lipid nanoparticle, transporter protein, modified silica nanoparticle, magnetic nanoparticle, and the like. For a review of drug delivery vectors, see Goyal P, Goyal K, Vijaya Kumar SG, Singh A, Katare OP, and Mishra DN; Liposomal drug delivery systems—clinical applications; Acta Pharm. March 2005; 55(1):1-25, which is incorporated herein by reference.

The inventors envision that the targeting moiety can be any molecular recognition entity, such as an antibody, aptamer, transporter protein, ligand, receptor, or a fragment thereof. For example, transferrin may be used to target cells that express the transferring receptor. Table 1 depicts several tumor cell recognition elements, which can be targeted by the targeting moiety in the practice of this invention. The skilled artisan, in the practice of this invention, may substitute any number of targeting moieties to achieve their particular zinc-mediated therapeutic goal.

TABLE 1
Tumor antigens as targets

| Types of Cancer | Expressed Antigens |
|---|---|
| Breast | Her-2 (human epithelial growth factor |
| | receptor type 2) |
| | Ep-CAM (epithelial cell adhesion molecule) |
| Prostate | PSCA, Sigma Receptor, CD-44, Transferrin |
| | Receptor |
| Liver | |
| Colon and Rectum | Ep-CAM (epithelial cell adhesion molecule) |
| | Transferrin Receptor |
| | Carcinoembryonic antigen (CEA) |
| Urinary Bladder | |
| Non-Hodgkin Lymphoma | |
| Melanomas of the skin | Mart-1, GM-2 |
| Kidney and Renal Pelvis | |
| Pancreas | Mesothelin |
| Oral Cavity and Pharynx | |
| Ovary | Folate Receptor, CA-125, Mesothelin |

### EXAMPLE 1

### Effects of Zinc on PC12 Cells

The addition of zinc to PC12 cells in vitro leads to time and dose-dependent cell death. This occurs with either undifferentiated or differentiated PC12 cells. In both cases, within 5 hours, cell death was accompanied by the progression to a more rounded morphology, shrinkage in overall cell size, and membrane blebbing, which is indicative of apoptosis. With the higher zinc concentrations (<100 μM), or longer time periods, significant cell lysis is also observed, which is indicative of necrosis, a novel observation for zinc .

When the PC12 cells were preincubated with the MEK inhibitor, U1026, zinc-induced cell death was greatly attenuated. Under those conditions, zinc cytotoxicity occurred with a delayed time-course, requiring overnight incubations to observe significant cell death. Under such circumstances, a low percentage of the cell populations underwent morphological changes associated with cell death, with cell lysis (an indicator of necrosis) predominating. Preincubation of these cells with inhibitors of the other members of the MAP kinase signaling cascades had no effect on zinc-mediated cell death. The data suggest that ERK mediates the cytotoxic effect of zinc, while JNK and p38 are not involved. Likewise, zinc treatment of PC12 cells induced ERK activation, while preincubation of cells with U1026 attenuated that activation. pERK could be detected within five minutes of treatment and remained at that level shown for at least five hours, which was the longest time period examined in this example.

These cytotoxic effects observed in PC12 cells (supra) are attributed to zinc. While ZnCl₂ was used in initial experiments, ZnSO4 was equally effective as ZnCl₂. No cell death, neither apoptosis nor necrosis, was observed when incubations include NiCl₂, CoCl₂ or CuSO₄ at the same range of the concentrations as zinc. Interestingly, Cd treatment did result in cell death, but through an ERK-independent mechanism. High (mM) concentrations of Mg, Ca, Mn or Li had no effect on cell viability. Similar experiments with IIC9 cells produced the same results.

### EXAMPLE 2

### Increased Intracellular Zinc Induces Cell Death In Multiple Cell Types

Exemplary cell types were treated in vitro with varying amounts of zinc (zinc chloride or zinc sulfate), in the presence or absence of the zinc ionophore pyrithione. As is depicted in tables 2 and 3, lower concentrations of zinc are effective in killing cells when the zinc ionophore is present, indicating that zinc function intracellularly. Cell death involves both apoptosis and necrosis as determined by morphological observation.

TABLE 2
Zinc-induced death of RAW (macrophage) cells

| | Treatment | | | | |
|---|---|---|---|---|---|
| Hours of | 25 μM | 25 μM + | 50 μM | 50 μM + | 100 μM |
| Incubation | zinc | pyr | zinc | pyr | zinc |
| 0.0 | 0.0 | 0.0 | | | |
| 1.0 | 0.0 | 0.0 | | | |
| 3.0 | 0.0 | 15.0 | | | |
| 5.0 | 0.0 | 35.0 | 0.0 | 50.0 | |
| 18.0 | | | 20.0 | | 100.0 |

pyr = pyrithione (zinc ionophore); numbers = % cell death

TABLE 3
Zinc-induced death of N18TG2 (neuroblastoma) cells
24 h incubation

| | |
|---|---|
| Treatment | % death |

| | | |
|---|---|---|
| 10 | μM zinc + pyr | 86 |
| 25 | μM zinc | 19 |
| 25 | μM zinc + pyr | 87 |
| 50 | μM zinc | 75 |
| 75 | μM zinc | 84 |
| 150 | μM zinc | 90 |

### EXAMPLE 3

### Zinc Kills IIC9 Cells in an ERK-Dependent Manner

In this example and the following examples, we show that zinc, (as in other cell lines such as PC12, RAW macrophages, and N18TG2 neuroblastoma cells) induces the apoptotic death of IIC9 embryonic fibroblasts. As with PC12 cells, death is ERK-dependent. Unlike previous examples, however, IIC9 cells are p53 minus. Thus zinc-mediated apoptosis occurs via a p53-independent mechanism. Death is also facilitated by the presence of the zinc ionophore, pyrithione, indicating that intracellular zinc initiates the death response. To understand the upstream activator of zinc-induced ERK1/2 activation, we examined the role of Ras in this process. Expression of dn-(dominant negative) Ras attenuates ERK1/2 activation by zinc and correspondingly reduces its cytotoxic effects. Raf-RBD pull-down experiments were used to confirm that zinc treatment activates Ras and to identify the specific Ras isoform that is activated. Importantly, zinc uniquely activates H-Ras. This contrasts the activation of K- and N-Ras that occurs when IIC9 cells are stimulated by mitogen. Thus, although the kinetics of activation of the Ras/ERK pathway are similar to those seen when induced by mitogen, the distinguishing feature appears to be the isoform specificity of Ras activation.

Cells and Incubation Conditions: IIC9 cells were grown in DMEM containing 10% FCS, P/S. Cells were plated in 12-well plates and grown to approximately 70% confluency. At that time, cells were serum arrested by changing the medium to DMEM (low glucose) without FCS. After 48 hours, cells were treated with zinc as described in the text. Cell death was quantified by trypan blue uptake. Cells were incubated with trypan blue (0.04%) for 5 min. Cells were examined microscopically and the number of blue cells and total cells were counted. Four fields were examined for each condition, with a minimum of 100 cells/field. To examine the effects of selective inhibitors, cells were preincubated with the indicated compound for 30 min. prior to zinc treatment.

Assays: ERK1/2 activation was determined by harvesting cells directly in boiling SDS-PAGE sample buffer and probing corresponding Western Blots with anti-pERK antibody (Cell Signaling). Blots were stripped and reprobed for total ERK (Cell Signaling). Ras activation was determined using the Raf-RBD assay (21,24). Identification of the specific Ras isoform activated was determined by probing the western blot with isoform-specific antibody (Upstate Biotechnology). Transfection of IIC9 cells was carried out using Novafector. Transfection efficiency of these cells is approximately 90%. The constructs for expression of dn-Ras and Bcl-Xl were kindly provided by Dr. J. Corbett and Dr. Chinnaduri (SLU).

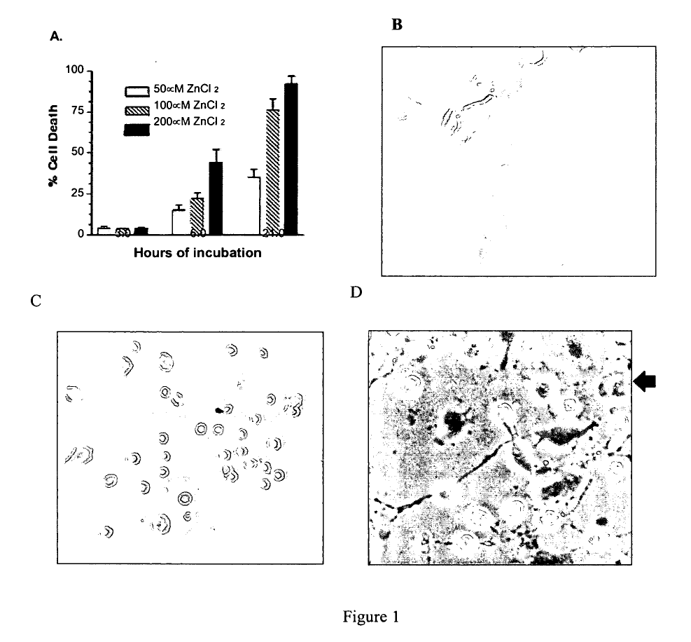

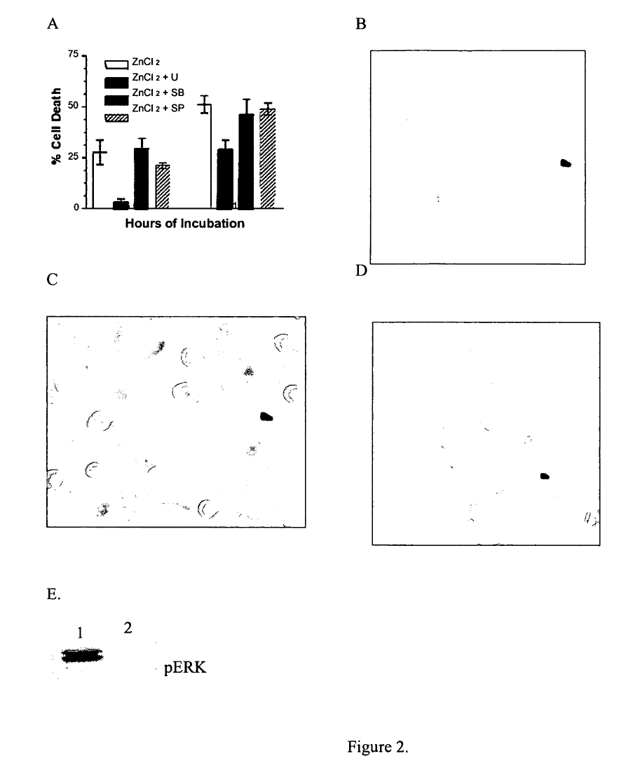

The addition of zinc to IIC9 cells led to cell death in a time- and concentration-dependent manner. Little or no death was observed during the first few hours of cell incubation with zinc concentrations as high as 200 μM (FIG. 1A). However, during this time frame, zinc does induce dramatic changes in cell morphology. In response to either 100 or 200 uM zinc, all cells were rounded and underwent differing degrees of cell shrinkage (FIG. 1C). This contrasts their normal, irregular, and somewhat elongated morphology (FIG. 1B) In response to 50 μM zinc , the lowest concentration used in this particular experiment, approximately 50% of the cell population display a rounded morphology at this time. After a 6 h period, cell death is evident, with the percentage of non-viable cells being relative to the concentration of zinc present (FIG. 1A). In this experiment, cell death was approximately 45%, 25%, and 15% when cells were treated with 200 μM, 100 μM and 50 μM zinc for 6 h respectively. Lysed cells could also be seen as a result of treatment with the higher zinc concentrations (FIG. 2D). With longer incubation times, the percentage of non-viable, cells continued to increase (FIG. 1B) and cell lysis became more predominant even at the lower zinc concentrations.

The effects observed are specific for zinc. ZnSO₄ is equally effective as ZnCl₂, indicating that zinc is the effective component. Treatment of cells with NiCl₂, CoCl₂ or CuSO₄ at the same range of concentrations as zinc does not result in the cell death of IIC9 cells. High (mM) concentrations of Mg, Ca, Mn or Li have no effect on cell viability.

JNK and p38 are well known effectors of stress signals, with established roles in apoptosis. These two members of the MAPK family, however, are not mediators of zinc-induced cytotoxicity since preincubation of cells with SP600125 or SB203580, well characterized selective inhibitors of JNK and p38 respectively, does not affect zinc-mediated cell death (FIG. 2A). In contrast, preincubation of IIC9 cells with U0126, an inhibitor of ERK activation, inhibits zinc-induced cell death. In the experiment shown, a 6-h incubation with 50 μM zinc leads to the death of approximately 25% of the population. Little or no death results when this concentration of zinc is added to cells pretreated with U0126. FIG. 2B-D shows that the morphological changes induced by zinc are also limited in cells preincubated with U0126.

Because blocking ERK activation attenuates zinc-mediated cell death, we reasoned that zinc treatment results in ERK activation. This was demonstrated by incubating IIC9 cells with 50M zinc and determining the presence of pERK by Western blotting with anti-pERK antiserum (FIG. 2E). ERK activation is rapid, detected within 5 min of zinc addition and remains elevated for at least 6 h, the longest time period examined (data not shown). Consistent with its effect on zinc cytoxicity, U0126 significantly reduces ERK activation by zinc. In the experiment shown, some pERK could be detected with longer exposure of the blot. Thus, it is possible that the residual ERK activity accounts for the ability of zinc to kill cells when it is present for the more extensive time periods. Alternatively, zinc may induce an additional death-pathway that is not ERK dependent.

### EXAMPLE 4

### Zinc-Mediated Cell Death Occurs via Regulated Apoptosis and Necrosis

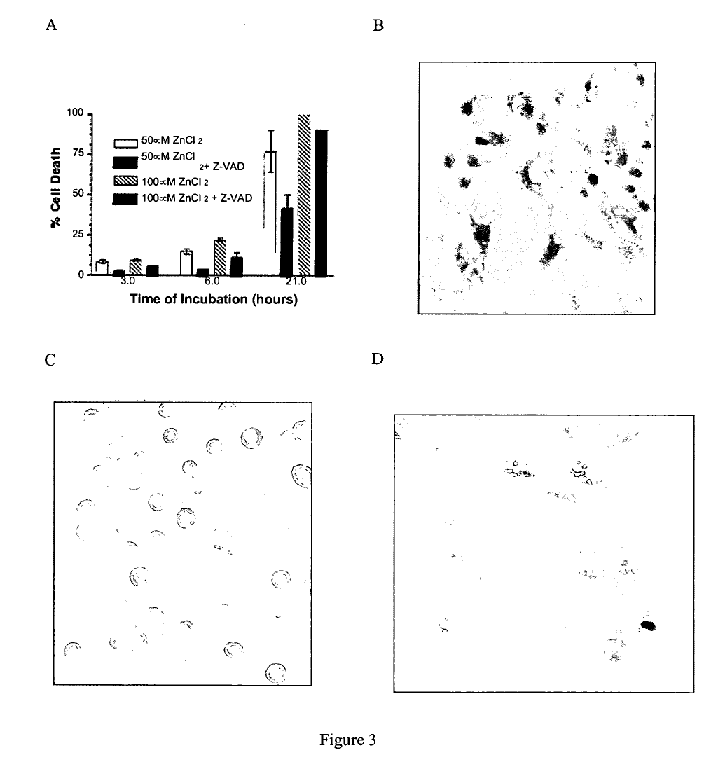

The initial morphological changes seen upon zinc addition are suggestive of apoptotic cell death (FIG. 1). To confirm the importance of the apoptotic pathway, we preincubated cells with Z-VAD, a general caspase inhibitor. As shown in FIG. 3A, addition of Z-VAD attenuated cell death. Cells treated with 50 μM zinc showed little cell death when pretreated with Z-VAD during the first 6 hours of zinc treatment. When cells are incubated with 100μM zinc, Z-VAD attenuates cell death by approximately 50 %. With longer time periods (21 h), the effect of Z-VAD was less dramatic. Cell death as a result of 50 μM zinc treatment was attenuated by 50% but limited attenuation was observed with 100 μM zinc. While not wishing to be bound by theory, those data can be explained by the possibility that Z-VAD does not completely inhibit caspases in IIC9 cells over this time period. Alternatively, zinc may induce both caspase-dependent (apoptotic) and caspase-independent (necrotic) cell death pathways, as suggested by the morphological changes that occur with the longer incubation times.

The processes involved in zinc-mediated cell death were further investigated by over-expressing Bcl-XL in IIC9 cells. Expression of Bcl-XL prevents zinc-mediated cell death even when IIC9 cells are incubated with concentrations of zinc as high as 200 μM (FIG. 3B-D). As shown, Bcl-XL expressing cells retain their normal morphology and do not undergo rounding, skrinkage, or other signs characteristic of apoptosis (FIG. 3D). This was true even after 24 h of treatment of IIC9 cells with 200μM zinc. Interestingly, over-expression of Bcl-Xl also eliminates any signs of cell lysis. This is a completely novel observation. The data indicate that over-expression of Bcl-XL prevents both the zinc-mediated apoptotic and necrotic pathways.

### EXAMPLE 5

### Mechanism of Erk Activation

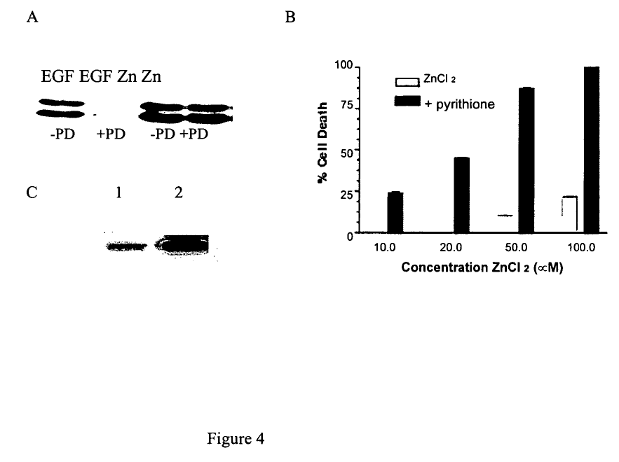

Previous studies have shown that zinc activates ERK1/2 in human epidermoid A431 cells and B82L parental fibroblasts in a manner that is blocked by inhibition of the EGF R and/or c-Src(45). In those cases, it appears that zinc stimulates a c-Src dependent phosphorylation of the EGF R. This trans-activation leads to enhanced ERK activity, and in the case of B82L cells, enhanced production of inflammatory cytokines. The effects of zinc on cell viability were undetermined. In contrast to those observations, treatment of IIC9 cells with PD153035 (EFG R inhibitor) does not alter ERK activation by zinc (FIG. 4A). Inhibition of the PDGF R or treatment of IIC9 cells with PP2 to inhibit Src does not inhibit zinc activation of ERK1/2 (data not shown). Extracellular zinc has been shown to activate ERK via a cell surface “zinc-sensing” receptor(3) To gain insight into the likelihood that zinc functions at the level of a cell surface receptor, we compared the effects of zinc treatment in the absence or presence of the heavy metal ionophore, pyrithione. In the presence of pyrithione, ERK activation is observed at markedly lower zinc concentrations than in its absence (FIG. 4B). Consistent with the enhanced ability of zinc to activate ERK when pyrithione is present, cell death also occurs at lower zinc concentrations (FIG. 4C). These data suggest that the immediate target for zinc-activation of the ERK1/2 signaling cascade that initiates a cell death response is intracellular.

Since zinc functions intracellularly, the time and concentration dependence of cell death seen in our experiments likely reflect the conditions necessary for cells to accumulate sufficient levels of zinc to induce the death pathway. For IIC9 cells, extracellular zinc concentrations of 50 μM or higher are necessary to do so, in the absence of pyrithione. Such levels are found in a number of tissues e.g. prostate, brain, pancreas (2,9,12,19,30) In brain, when conditions of excitotoxicity lead to sustained release of zinc from a subset of glutamenergic neurons, extracellular concentrations higher than 300 μM have been reported. Under such circumstances, transport of zinc into the post-synaptic cell is associated with its death (2,9).

### EXAMPLE 6

### Zinc Activate ERK via a Specific Ras

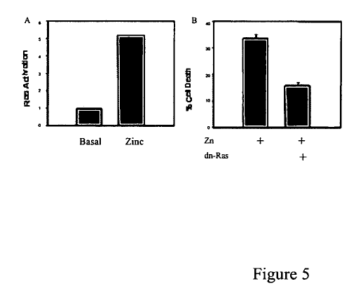

Because Ras is a common upstream activator of ERK1/2 (36), we reasoned that zinc may induce the phosphorylation of ERK1/2 via Ras. This was examined by incubating IIC9 cells with 100 uM zinc and assessing Ras activation by RBD pull-down of GTP-Ras followed by Western blot analysis with a pan-Ras antibody. The results show that zinc induces approximately a 5-fold activation of Ras (FIG. 5A). The critical role of Ras in mediating the effects of zinc was confirmed by examining the effect of over-expression of dnRas. Expression of dn-Ras inhibits ERK activation, indicating that the Ras-Mek pathway is the predominant mechanism used by zinc to activate ERK. Consistent with Ras as the upstream activator of the cytotoxic effect of zinc on IIC9, expression of dn H-Ras also attenuates zinc-mediated cell death (FIG. 5B).

IIC9 cells express H-, N- and K-Ras. Previously we found that stimulation of IIC9 cells with thrombin results in an approximate 3- to 5-fold activation of Ras. Our more recent studies show that thrombin induces a rapid activation of N-Ras, followed by a sustained activation of K-Ras. Because thrombin and zinc activation of Ras in IIC9 cells result in different biological outcomes, we asked whether they activate distinct Ras isoforms. In contrast to the results obtained when cells are stimulated with thrombin, zinc stimulation results in the selective activation of H-Ras.

### EXAMPLE 7

### Production of Liposomes Containing Zinc

To ensure delivery of relatively high concentrations of zinc to tumor cells, we developed a targeted encapsulated liposome delivery method. Liposomes, spherical, self-enclosed vesicles composed of amphipathic lipids, have been widely studied and are employed as vehicles for in vivo administration of therapeutic agents. In particular, the so-called long circulating liposomes formulations which avoid uptake by the organs of the mononuclear phagocyte system, primarily the liver and spleen, have found commercial applicability. This method has several advantages: 1) it allows for the delivery of high therapeutic concentrations of zinc, 2) only the targeted cells are exposed to high zinc concentrations. This effectively should increase the therapeutic range of zinc and finally 3) by encapsulating zinc within long circulating liposomes formulations containing polyethyleneglycol (PEG-liposome) the effective half life of the zinc can be effectively increased.

The first issue concerning the encapsulation of high concentrations of zinc was addressed by employing an ethanol injection method employing a complex mixture of neutral and positively charged phospholipids plus cholesterol (infra). The inventor envisions that liposomes may contain polyethylene glycol (PEG).

The second issue is to devise a method to achieve site specific delivery of the zinc containing liposomes. We choose to employ an approach that utilizes targeting ligands. The advantage of this method is that the method can be easily adapted to target different types of cells by altering the targeting ligand. For example, an antibody can be attached to the liposomes' surfaces. We choose an approach in which the targeting ligand is attached to the free ends of the polymer chains forming the surface coat on the liposomes (Allen. T. M., et al., Biochim. Biophys. Acta, 1237:99-108 (1995); Blume, G., et al., Biochim. Biophys. Acta, 1149:180-184 (1993)).

We choose the prostate cancer cell line PC3 cells to demonstrate that therapeutic concentrations of zinc (“killing”) can be delivered to cancer cells. These cells express high levels of the transferrin receptor and can be targeted by covalently attaching transferrin to the formed liposomes.

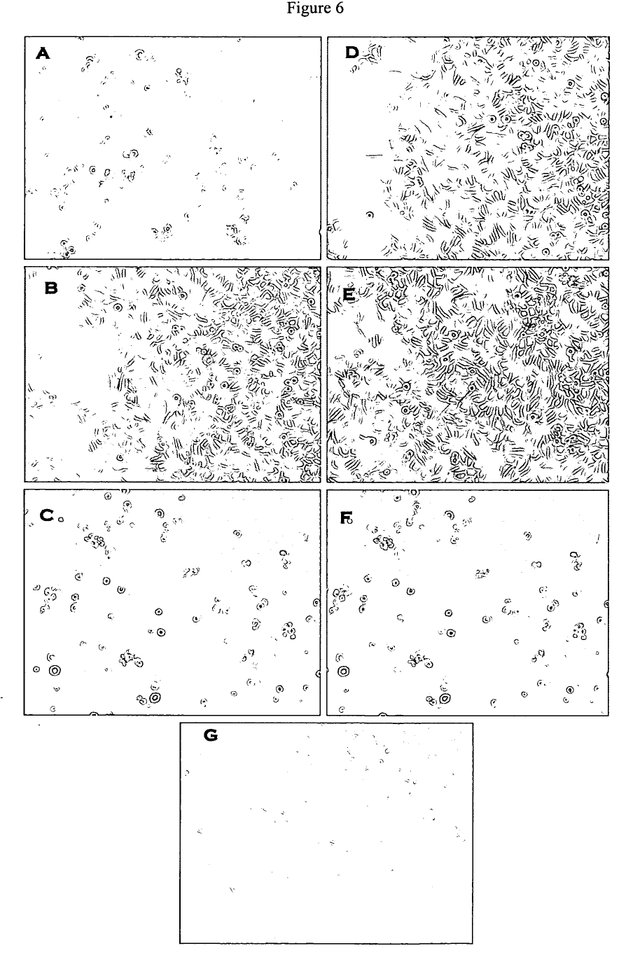

We next tested the ability of these liposomes to kill PC3 cells in culture (FIG. 6). Treatment of PC3 cells with liposomes that contained high concentrations of zinc and were covalently linked to transferrin resulted in effective cell death. In contrast, liposomes that either did not encapsulate zinc or did not contain transferrin were ineffective (FIG. 6).

In a prophetic liposome, PEG is incorporated into our liposomes to ensure that our liposome preparations have reduced opsinisation in the plasma. Thus, the liposomes exhibit reduced uptake by the reticuloendothelial system (RES), which primarily consists of macrophages in the liver and the spleen. Furthermore, these liposomes can preferentially accumulate in solid tumors via the increased permeability of the tumor endothelium and reduced lymphatic drainage, or enhanced permeability and retention (EPR) effect.

Non-prophetically, zinc containing liposomes were prepared by injecting ethanolic solutions of lipids into 10 mM Hepes buffer pH 6 containing 2.5 M ZnCl₂. To make the ethanolic lipid solution, lipids initially dissolved in chloroform were dried by passing gaseous nitrogen over the solution. The dried lipids were dissolved in ethanol at a concentration of 2 to 50 pmol of phospholipids per mL of alcohol. The alcoholic lipid solution containing dioleoyl phosphatidyly choline, 1,2 dioleoyl phosphatidyly ethanolamine, 1,2 dioleolyl phosphatidyletanolamine-N-4-(p-maleimido-phenyl)butyramide) and cholesterol in a ratio of 7:2:0.05:1 was slowly (0.5 ml/min) injected through a Hamilton syringe into 3 mL of a magnetically stirred Hepes buffer, containing 2.5 mM ZnCl₂ The final concentration of ethanol was between 2.5-7.5%. During injection of the lipids, the solution was constantly stirred by a standard magnetic stirrer. To ensure that the temperature of the liposome solution was well above the phase transition of the lipids, the temperature was kept at 350 C. Because the transferrin coupling reaction is carried out in sodium borate buffer pH 8.5, the liposomes were dialyzed overnight against a 0.15 mM sodium borate, 0.1 mM EDTA buffer pH 8.5. Before coupling transferrin to the liposomes, 100 μg of transferrin was thiolated by treating transferrin with 400 nmoles of fresh Traut's reagent in 2 ml of the sodium borate buffer for 1 hour under dark conditions at room temp. To link the thiolated transferrin to the liposomes, thiolated transferrin was immediately added to the liposomes and left for 24 hrs at 37° C. After 24 hours the liposomes were dialyzed against 50 mM Tris buffer pH 7.2 and then briefly sonicated in a bath sonifier, for approximately 5 minutes.

Although several embodiments have been described in detail for purposes of illustration, various modifications may be made to each without departing from the scope and spirit of the invention. Accordingly, the invention is not to be limited, except as by the appended claims.

1. A method of killing a cell, comprising the steps of (a) contacting a cell with a therapeutically effective amount of a zinc composition, wherein the zinc composition comprises a delivery vector and a zinc compound, and (b) allowing the zinc compound to move into the cell, wherein the cell undergoes cell death.

2. The method described in claim 1 wherein cell death is selected from the group consisting of apoptosis, necrosis, and a combination of apoptosis and necrosis.

3. The method described in claim 1 wherein cell death is by caspase-independent apoptosis.

4. The method described in claim 1 wherein cell death is by p53-independent apoptosis.

5. The method described in claim 1 wherein cell death is by necrosis.

6. The method described in claim 1 wherein cell death is by ERK-dependent necrosis.

7. The method described in claim 1 wherein cell death is by p53-independent apoptosis and necrosis.

8. The method described in claim 1 wherein the cell is a disease cell which can undergo unchecked cell proliferation.

9. The method described in claim 8 wherein the cell is a cancer cell.

10. The method described in claim 9 wherein the cancer cell is in a patient.

11. The method described in claim 9 wherein the cancer cell is a prostate cancer cell.

12. The method described in claim 9 wherein the cancer cell is a PC3 cell.

13. The method described in claim 1 wherein the delivery vector is selected from a group consisting of a liposome, a nanocarrier, a colloidal gold particle, a peptide, a virus particle, an intracellular bacteria, a vesicle, a cyclodextrin microsphere, a lipid nanoparticle, a transporter protein, a modified silica nanoparticle, and a magnetic nanoparticle.

14. The method described in claim 13 wherein the delivery vector is a liposome.

15. The method described in claim 13 wherein the delivery vector is a transporter protein.

16. The method described in claim 15 wherein the transporter protein is a transferrin.

17. The method described in claim 1 wherein the delivery vector contains a targeting moiety.

18. The method described in claim 1 wherein the delivery vector is a liposome which contains a targeting moiety.

19. The method described in claim 17 wherein the targeting moiety is an antibody directed to a cancer-specific cell-surface marker.

20. The method described in claim 18 wherein the targeting moiety is an antibody directed to a cancer-specific cell-surface marker.

21. The method described in claim 19 wherein the cancer-specific cell-surface marker is selected from the group consisting of human epithelial growth factor receptor type 2 (“Her-2”), epithelial cell adhesion molecule (“Ep-CAM”), carcinoembryonic antigen (“CEA”), folate receptor, CA-125, mesothelin, PSCA, sigma receptor, CD-44, transferrin receptor, Mart-1, and GM-2.

22. The method described in claim 20 wherein the cancer-specific cell-surface marker is selected from the group consisting of human epithelial growth factor receptor type 2 (“Her-2”), epithelial cell adhesion molecule (“Ep-CAM”), carcinoembryonic antigen (“CEA”), folate receptor, CA-125, mesothelin, PSCA, sigma receptor, CD-44, transferrin receptor, Mart-1, and GM-2.

23. The method described in claim 19 wherein the cancer-specific cell-surface marker is PSCA and the cell is a prostate cancer cell.

24. The method described in claim 20 wherein the cancer-specific cell-surface marker is PSCA and the cell is a prostate cancer cell.

25. The method described in claim 1 wherein (a) the zinc composition is administered to the cell via a delivery means selected from the group consisting of intravenous, oral, intratumoral, local topical application, intranasal, inhalation, and intraperitoneal.

26. The method described in claim 1 wherein (a) the zinc composition is administered to the cell via local topical application to a tumor.

27. The method described in claim 1 wherein the zinc compound is zinc chloride.

28-68. (canceled)
